(19) United States
(12) Patent Application Publication
(10) Pub. No.: US 2026/0083950 A1
(43) Pub. Date: Mar. 26, 2026

(54) LOCATOR AND GUIDE FOR NEEDLE

(71) Applicant: Cochlear Limited, Macquarie University, NSW (AU)

(72) Inventors: Wim BERVOETS, Wilrijk (BE); Daniel SMYTH, Dublin (IE); Wolfram Frederik DUECK, Berlin (DE); Jessica BRAND, Mechelen (BE)

(21) Appl. No.: 19/109,387

(22) PCT Filed: Sep. 1, 2023
(86) PCT No.: PCT/IB2023/058682

§ 371 Date: Mar. 6, 2025

Related U.S. Application Data
(60) Provisional application No. 63/406,015, filed on Sep. 13, 2022.

(51) Int. Cl. A61M 39/02

(52) U.S. Cl. CPC A61M 39/0208; A61M 2039/0205; A61M 2039/0238; A61M 2210/0662

(57) ABSTRACT

Presented herein are techniques for locating a target structure disposed in a body of a subject and/or guiding a needle with respect to the target structure. In one example, an apparatus is provided that includes a locator portion and a needle guide portion. The locator portion is configured to locate a target structure disposed in a body of a subject, and a needle guide portion defines an aperture configured to have a needle positioned therewith. The needle guide portion is configured to align the needle at a target angle relative to the target structure.

### BACKGROUND

### Field of the Invention

The present invention relates generally to location of a target structure disposed in a body of a subject and/or guidance of a needle with respect to the target structure.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, an apparatus is provided. The apparatus comprises: a locator portion configured to locate a target structure disposed in a body of a subject; and a needle guide portion defining an aperture configured to have a needle positioned therewith, wherein the needle guide portion is configured to align the needle at a target angle relative to the target structure.

In another aspect, another apparatus is provided. The other apparatus comprises: a base portion configured to mate with a subcutaneous structure; and a handle portion that is secured to, and elevated from, the base portion, wherein the handle portion is configured to allow a needle to pass there through to the subcutaneous structure.

In another aspect, a method is provided. The method comprises: mating a base portion of a needle control device with a subcutaneous reservoir in a subject; positioning a needle with a needle guide portion of the needle control device to align the needle at a target angle relative to the subject; and while the needle is aligned at the target angle relative to the subject, administering an injection via the needle into the subcutaneous reservoir.

In another aspect, another method is provided. The method comprises: mating a base portion of a locator apparatus with a subcutaneous structure in a subject; positioning a marking device with a guide portion of the locator apparatus; and with the marking device, marking a position on the subject where the subcutaneous structure is located.

### BRIEF DESCRIPTION OF THE DRAWINGS

Embodiments of the present invention are described herein in conjunction with the accompanying drawings, in which:

FIG. 1 is a schematic diagram illustrating a system with which aspects of the techniques presented herein can be implemented;

FIGS. 2A and 2B illustrate a locator and needle guide in operation, in accordance with certain embodiments presented herein;

FIGS. 3A-3D illustrate respective embodiments of a locator and needle guide that enable varied insertion points of a needle into a subcutaneous structure, in accordance with certain embodiments presented herein;

FIG. 4 illustrates a cross-section of a needle/syringe and needle guide portion that are uniquely compatible with each other, in accordance with certain embodiments presented herein;

FIG. 5 is a flowchart of a method, in accordance with certain embodiments presented herein; and

FIG. 6 is a flowchart of another method, in accordance with certain embodiments presented herein.

### DETAILED DESCRIPTION

There are a number of different types of devices that deliver therapeutic substances (drugs) to a recipient. For example, the inner ear can receive medical treatment/therapy via therapeutic substances administered through a cochlear implant to pass the blood level barrier. The therapeutic substances can be administered into a subcutaneous reservoir/port that is implanted at the surface of the skull and in contact with a cochlear electrode. Once implanted, the subcutaneous reservoir typically requires refill cycles to sustain the therapy, potentially based on different therapeutic substance types. The refills can occur frequently throughout the lifetime of the implant and/or patient (e.g., months or years). As a result, many patients and medical professionals (e.g., surgeon, clinician, etc.) or other persons administering the therapeutic substance are subjected to frequent refill operations over a long period of time.

During a refill cycle, a medical professional can administer the therapeutic substance(s) by inserting a needle through a septum of the subcutaneous reservoir and injecting the therapeutic substance, via the needle, using a syringe. Before injection, the medical professional locates the subcutaneous reservoir, typically using palpation. However, locating the subcutaneous reservoir using only palpation can require undue trial-and-error and can damage one or more subcutaneously implanted portions (e.g., the septum) and/or the needle. As a result, palpation-based location can be a time-consuming process, the outcome of which ultimately depends on the skill of the medical professional. Furthermore, the needle can potentially be inserted at a wide range of angles and if the insertion angle is too small/extreme, the needle can miss the septum, causing the therapeutic substance to be injected at the wrong location and/or damage to the septum. This can lead to a host of issues, such as discomfort and/or harm to the patient, damage to the needle and/or subcutaneous reservoir, etc.

Increasing the size of the septum to mitigate these issues can create other problems. Larger septa tend to induce patients to tap or otherwise fiddle with the subcutaneous reservoir, which can introduce unwanted acoustic signals into the cochlea and/or create over-pressure that potentially damages the subcutaneous reservoir, which in turn risks harm to an implanted device and/or cochlea. In addition, the larger the septum, the more limited the locations in the subject where the septum can be implanted.

Accordingly, to improve patient care/after-care/follow-up, techniques are provided herein for a locator and needle guide. The locator and needle guide can allow the person administering the injection (e.g., clinician) to quickly locate the subcutaneous septum without risking damage thereto. Using the techniques presented herein, even relatively small (e.g., 3.5 mm diameter or less) subcutaneous septa can be quickly located, which can allow for wider use of smaller septa with the benefits of less patient interference and enable implantation at a wider range of locations (e.g., close to the pinna).

In addition to locating the subcutaneous reservoir/septum, the locator and needle guide presented herein can also provide angular orientation/guidance of the needle. More specifically, the intrinsic design functionality of the locator and needle guide can enable a highly accurate needle-to-septum alignment. For example, the locator and needle guide can ensure an orthogonal injection angle. The locator and needle guide can allow for a refill process that is smooth and comfortable for the patient. Using the locator and needle guide, the refill process can be performed by any suitable caretaker, even one who is not a medical professional. The locator and needle guide can include a handle configure to enable the person performing the refill to manipulate/use the instrument without harm, especially when inserting the needle.

In some embodiments, the locator and needle guide can be magnet-less. For example, the locator and needle guide can be constructed and used without any ferro-magnetic materials. Because, in some implementations, no magnetic and/or ferro-magnetic materials are applied to establish locator and guiding functionality, there is no additional risk for the patient when undergoing certain medical procedures (e.g., Magnetic Resonance Imaging (MRI) procedures).

Merely for ease of description, the techniques presented herein are primarily described with reference to a specific implantable medical device system, namely a cochlear implant system. However, it is to be appreciated that the techniques presented herein can also be partially or fully implemented by other types of implantable medical devices. For example, the techniques presented herein can be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, therapeutic substance delivery systems, defibrillators, functional electrical stimulation devices, catheters, seizure devices (e.g., devices for monitoring and/or treating epileptic events), sleep apnea devices, electroporation devices, etc.

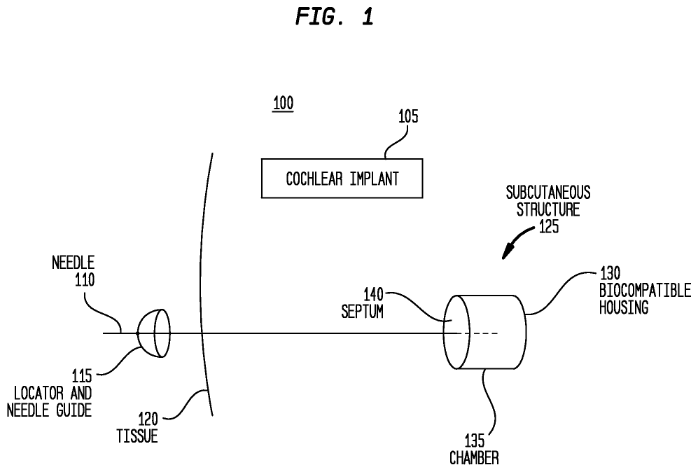

FIG. 1 illustrates an example system 100 in which aspects of the techniques presented herein can be implemented. System 100 includes a cochlear implant 105, needle 110, and locator and needle guide 115. Locator and needle guide 115 can be made of a rigid and/or semi-rigid material (e.g., metal, plastic, silicone, etc.), potentially in combination with flexible portions. Tissue (e.g., skin, muscle, fat, etc.) 120 of a recipient are also shown for reference. Cochlear implant 105 is configured to be implanted under tissue 120 in the recipient. Cochlear implant 105 can be configured to electrically stimulate the recipient's auditory nerve cells based on received sound signals, bypassing absent or defective hair cells that normally transduce acoustic vibrations into neural activity, in a manner that causes the recipient to perceive one or more components of the received sound signals.

Subcutaneous structure (e.g., a subcutaneous reservoir such as a therapeutic substance delivery reservoir, a refill port, etc.) 125 includes a biocompatible housing 130 defining at least one chamber 135 configured to have therapeutic substance disposed therein. The at least one chamber 135 includes a skin-facing layer (e.g., membrane, septum, etc.) 140 through which the therapeutic substance can be injected into the at least one chamber 135.

In this example, a person (e.g., clinician) can be attempting to administer an injection (e.g., a therapeutic substance) via needle 110 into subcutaneous structure 125. The injection can be part of a long-lasting therapy process after an initial surgery (e.g., during which a surgeon can implant subcutaneous structure 125). The person (who can know the general location of subcutaneous structure 125) can use locator and needle guide 115 to accurately locate subcutaneous structure 125 and physically align needle 110 by physically interfacing with the external profile of implanted reservoir. The person may use locator and needle guide 115 to push down tissue 120 around subcutaneous structure 125. When it has a corresponding shape to subcutaneous structure 125 (e.g., at least one mating portion), locator and needle guide 115 may thereby align with subcutaneous structure 125. In some examples, an internal portion of locator and needle guide 115 may allow the local skin to remain uncompressed (in contrast to the skin near a circumference/edge of locator and needle guide 115).

Locator and needle guide 115 can align needle 110 at a target angle (e.g., a target entry angle) with respect to subcutaneous structure 125 and/or the skin of the subject. In one example, the target angle can be approximately orthogonal/perpendicular (i.e., 90 degrees) with respect to septum 140. Acceptable target angle ranges can be within 5, 10, or 20 degrees of 90 degrees. Thus, the target angle can be between 85 and 95 degrees, 80 and 100 degrees, or 70 and 110 degrees. It will be appreciated these ranges are provided as examples, and that the target angle can be any suitable angle, such as an angle outside the range of 70 to 110 degrees.

Locator and needle guide 115 can direct needle 110 into septum 140 to replenish/fill chamber 135 with a therapeutic substance. In one example, subcutaneous structure 125 can have a hard (e.g., titanium) bottom portion configured to prevent needle 110 from piercing/damaging biocompatible housing 130 and potentially injuring the patient. The hard bottom portion can also act as tactile confirmation (after an initial, slight mechanical resistance followed by breakthrough caused by piercing septum 140) that subcutaneous structure 125 has been reached. Once needle 110 has been inserted via locator and needle guide 115 into subcutaneous structure 125, the person administering the therapeutic substance can inject the therapeutic substance via a syringe connected to needle 110 (not shown). In some examples, needle 110 may be a double-barrel needle. For instance, needle 110 may refill chamber 135 (with assistance from locator and needle guide 115) by inject a fresh/new therapeutic substance the remaining fluid in chamber 135 while carrying any previously injected therapeutic substance from chamber 135.

Locator and needle guide 115 can facilitate repeatable injection of therapeutic substances into subcutaneous structure 125 by locating it and controlling the angle of insertion of needle 110. Due to the intuitive and non-penetrating nature of locator and needle guide 115, it can improve the safety of the injection process while reducing the chances of causing unnecessary pain or discomfort to the subject in the event of multiple injection attempts due to inaccurate initial needle placement/insertion. Locator and needle guide 115 can be operable without relying on active navigation and generated location fields from subcutaneous structure 125 (e.g., implant electronics/magnets). Depending on the nature and design of subcutaneous structure 125, locator and needle guide 115 can help simplify administration of therapeutic substances, particularly if subcutaneous structure 125 is small and/or situated in a location where needle 110 is difficult to maneuver.

In the example of system 100, locator and needle guide 115 can be used to locate subcutaneous structure 125 (coupled to cochlear implant 105) and align needle 110 for repetitive therapeutic substance administration through the skin; however, it will be appreciated that locator and needle guide 115 can be used in applications that involve any suitable target structure disposed in a body of a subject. In one example, locator and needle guide 115 can be used in applications that do not involve a subcutaneous structure. For instance, locator and needle guide 115 can be used to assist in accurately locating a needle relative to an eyeball (or any other suitable target structure disposed in the body of the subject) when injecting a therapeutic substance into the eyeball.

It will be appreciated that locator and needle guide 115 can also be used for purposes/procedures other than refilling an implanted chamber. In one example, locator and needle guide 115 can be used for tattoo marking. For example, locator and needle guide 115 can be used to direct a marker or tattoo gun to place a permanent or semi-permanent marking on the skin over a subcutaneous septum. The marker therefore indicates the location of the subcutaneous septum for subsequent therapeutic substance administrations.

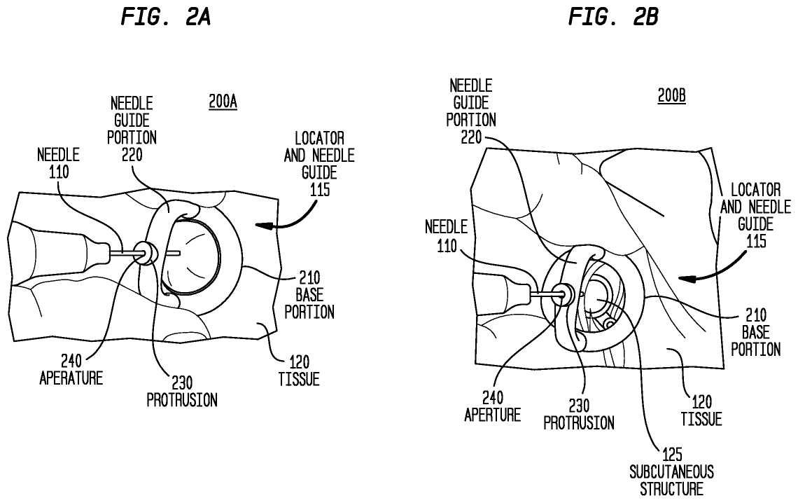

With continuing reference to FIG. 1, FIGS. 2A and 2B illustrate partial views 200A and 200B of system 100, in accordance with certain embodiments presented herein. View 200A shows locator and needle guide 115 positioned over tissue 120 covering subcutaneous structure 125. In view 200B, locator and needle guide 115 is positioned similarly, but tissue 120 is pulled back to reveal subcutaneous structure 125. It will be appreciated that, because locator and needle guide 115 generally avoids any need to expose subcutaneous structure 125, view 200A generally represents what would be seen by the clinician during actual use and view 200B is shown for illustrative purposes. FIGS. 2A and 2B will be discussed concurrently as follows.

As shown, locator and needle guide 115 includes base portion 210 and needle guide portion 220. Together, base portion 210 and needle guide portion 220 can axially align needle 110 with subcutaneous structure 125. For example, base portion 210 and needle guide portion 220 can orthogonally (e.g., at a 90-degree angle) align needle 110 with subcutaneous structure 125 for injection.

Base portion 210 is configured to mate with subcutaneous structure 125. Base portion 210 has a ring shape which allows base portion 210 to fit around the circular cross-section of subcutaneous structure 125 near tissue 120. Base portion 210 can be sized to ensure that needle guide portion 220 is aligned with subcutaneous structure 125 and needle 110 punctures septum 140 of subcutaneous structure 125. For example, the ring shape of base portion 210 can allow needle 110 to pass through base portion 210 into subcutaneous structure 125. Base portion 210 can also be referred to as a “locator portion” that is configured to locate a target structure (e.g., subcutaneous structure 125) disposed in a body of a subject.

Locator and needle guide 115 also includes needle guide portion 220, which is secured to, and elevated from, base portion 210. Needle guide portion 220 includes protrusion 230 which defines aperture 240. In this example, aperture 240 is centered and orthogonal with respect to needle guide portion 220. As shown, needle guide portion 220 is configured to allow needle 110 to pass there through to subcutaneous structure 125. More specifically, aperture 240 (defined by needle guide portion 220) is configured to have needle 110 inserted there through. Needle guide portion 220 can also be referred to as a “handle portion.” It will be appreciated that, while base portion 210 and needle guide portion 220 are integrated in locator and needle guide 115, some variations may involve physically separate/modular base and needle guide portions.

Needle guide portion 220 is configured to align needle 110, when inserted there through, at a target angle relative to the target structure (e.g., subcutaneous structure 125 and/or septum 140). In operation, a clinician (or any other person) can mate base portion 210 with subcutaneous structure 125. The clinician can further insert needle 110 into needle guide portion 220 to align needle 110 at the target angle relative to the subject. While needle 110 is aligned at the target angle, the clinician can administer an injection via needle 110 into subcutaneous structure 125.

In one example, the clinician can mate base portion 210 with the subcutaneous structure 125 by pressing base portion 210 to the subject. To prevent damage to tissue 120, base portion 210 can have an inner dimension configured to accommodate both subcutaneous structure 125 and tissue 120 when base portion 210 mates with subcutaneous structure 125. In one example, the inner dimension can be selected based on an average skin thickness. For example, the ring shape of base portion 210 can have an internal diameter equal to six millimeters greater than an outer diameter of subcutaneous structure 125. In this example, the inner diameter of base portion 210 may, when centered about subcutaneous structure 125, be offset by three millimeters from the outer diameter of subcutaneous structure 125. This controlled offset can allow tissue 120 to bend along the contour of subcutaneous structure 125 when the clinician axially pushes base portion 210 to the subject, thereby avoiding over-pressing (e.g., “pinching”) and potential damage of tissue 120. Thus, locator and needle guide 115 can be used even if the patient has thick skin flaps and the outer diameter of subcutaneous structure 125 is small.

In a further example, the inner dimension (e.g., shape, diameter, length, etc.) of base portion 210 can be adjustable. For instance, base portion 210 may have a standard design to accommodate a fixed range, and snap-on part(s) may be added to increase or decrease the diameter of base portion 210. Additionally/alternatively, snap-on part(s) may be added to increase or decrease one or more areas of the diameter (e.g., three or four studs may be equally distributed over the circumference). The base portion 210 may thereby accommodate subcutaneous structures of varying types/sizes. The base portion 210 may also/alternatively accommodate for thinner/thicker skin flap ranges as well.

This adaptability can allow the clinician to optimize the inner diameter to accommodate the skin thickness of a given patient, as skin thickness can vary from person to person. Thus, instead of having a fixed offset of three or six millimeters (for example) and an outer diameter of eight millimeters (for example), base portion 210 can have an adjustable inner diameter that can be set within a range of values (e.g., offset values between one and eight millimeters, or any other suitable range of values). Alternatively, a plurality of locator and needle guides can be provided, each having a base portion with a different fixed inner dimension, and the clinician can select the appropriate locator and needle guide (e.g., locator and needle guide 115) for use with a particular patient, given the patient's skin thickness and/or the outer dimension of subcutaneous structure 125.

It will be appreciated that base portion 210 can be shaped any suitable manner to be disposed around an outer dimension of subcutaneous structure 125. For example, if the outer dimension of subcutaneous structure 125 is an oval, then base portion 210 can be an oval; if the outer dimension of subcutaneous structure 125 is a square, then base portion can be a square. Base portion 210 may be shaped to include an additional offset, e.g., to account for skin thickness. In some examples, any suitable number of add-on parts/features (e.g., three or four) may adequately align the base portion 210 to subcutaneous structure 125.

Alternatively, a plurality of locator and needle guides can be provided, each having a base portion with a different shape, and the clinician can select the appropriate locator and needle guide (e.g., locator and needle guide 115) for use with a particular patient, given the shape of subcutaneous structure 125. The plurality of locator and needle guides may be disposable, reusable, or a combination thereof.

In another example, needle guide portion 220 can be elevated from base portion 210 by an adjustable/adaptable height. The clinician can thereby adjust the elevation of needle guide portion 220 from base portion 210, for example, before inserting needle 110. The adaptable height can be used to adjust the height of a stopper located in needle guide portion 220. For example, a modular portion may abut a rigid portion of the needle/syringe (e.g., protrusion 230 may be configured as a separate portion from locator and needle guide 115). The modular portion part may slide in and snap to needle guide portion 220 at specific heights; alternatively, a threading interface may be applied to adjust the height of protrusion 230. The stopper can act as a hard stop that prevents needle 110 from being inserted too far into subcutaneous structure 125, which could lead to damage of needle 110 and/or subcutaneous structure 125and could also cause particle formation in chamber 135 after multiple uses.

In another example, needle guide portions having various heights may be provided, or a standard design of needle guide portion 220 may be adjusted to a target height using a threading solution or break-off leg segments that fit into the base portion 210. In still another example, a tube/space (which may be cut to a specific length by a surgeon) may be added to the needle shaft. The amount that needle 110 is permitted to extend into subcutaneous structure 125 can depend on the thickness of the patient's skin. In one example, the clinician can measure the thickness of the patient's skin and, based on the thickness of the patient's skin, adjust the height of needle guide portion 220 such that the stopper is at a height that prevents needle 110 from extending beyond a threshold depth into subcutaneous structure 125.

It will be appreciated that locator and needle guide 115 can include just one (or neither) of an adjustable height feature or a stopper. For example, if locator and needle guide 115 has an adjustable height but not a stopper, the clinician can nonetheless use the adjustable height feature to set locator and needle guide 115 to a height that is most comfortable for the clinician to administer the injection and/or suits a specific needle length.

Locator and needle guide 115 has an “open design” that offers a full, direct view of the bulging skin to provide direct visual/tactile feedback to the clinician regarding the placement of locator and needle guide 115 relative to subcutaneous structure 125. In an alternative embodiment, locator and needle guide 115 can further include a flexible membrane (not shown) affixed to base portion 210 and/or needle guide portion 220. The flexible membrane, such as silicone, can be configured to adapt to an outer dimension of subcutaneous structure 125 when locator and needle guide 115 is pressed to subcutaneous structure 125 through tissue 120. Thus, the flexible membrane can permit the clinician to observe the bulging of subcutaneous structure 125 while preventing needle 110 from harming the clinician during insertion. In a further example, the flexible membrane can be transparent to provide further visibility.

Locator and needle guide 115 can minimize risk of harm to both the clinician and the patient. The clinician can manipulate locator and needle guide 115 with only two fingers, and the open design of locator and needle guide (and/or the flexible membrane) can provide visual feedback that subcutaneous structure 125 has been located while preventing needle 110 from injuring the clinician. Risk of skin damage to the patient using locator and needle guide 115 is minimal due to the controller/optimized diameter offset value (e.g., three millimeters), possibly in combination with a maximum protrusion height of subcutaneous structure 125 above the skull surface. Furthermore, by reducing or eliminating any need for palpation actions to locate subcutaneous structure 125, locator and needle guide 115 can also reduce or eliminate potential damage to tissue 120 and/or subcutaneous structure 125 resulting from the palpation action.

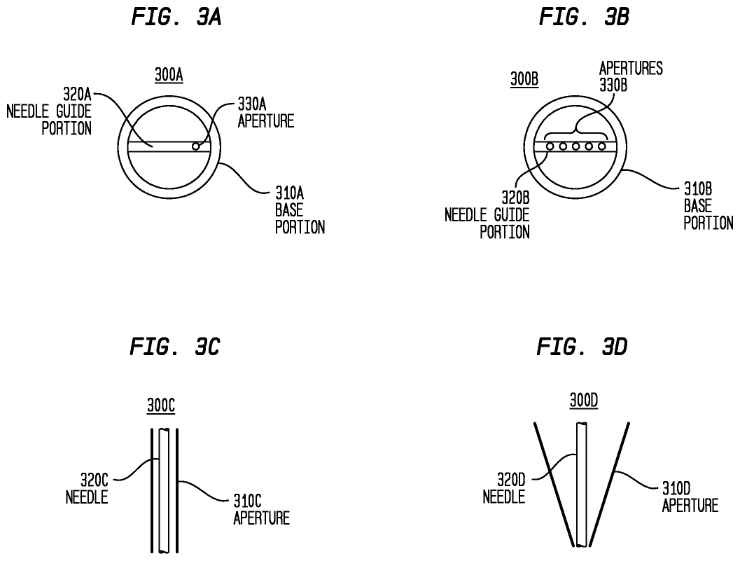

FIGS. 3A-3D illustrate respective embodiments of a locator and needle guide that enable varied insertion points of a needle into a subcutaneous structure, in accordance with certain embodiments presented herein. If the needle punctures the same spot in the septum of a subcutaneous structure multiple times, the lifetime of the septum can be shortened. As a result, the example embodiments shown in FIGS. 3A-3D are provided to cause needle to penetrate multiple varied insertion points in the same general area (e.g., the septum) and thereby maximizing the sealing longevity of the septum.

With reference first to FIG. 3A, shown is a top view of locator and needle guide 300A. Locator and needle guide 300A includes base portion 310A and needle guide portion 320A. Base portion 310A can be similar to base portion 210 (FIGS. 2A and 2B). Locator and needle guide 300A can be similar to locator and needle guide 115 (FIGS. 1, 2A, and 2B), except that needle guide portion 320A defines aperture 330A, which is off-center with respect to needle guide portion 320A.

In operation, the clinician (or any other person) can axially rotate locator and needle guide 300A (or a substantially identical locator and needle guide) with each subsequent injection. For example, the clinician can administer an initial injection with locator and needle guide 300A angled as shown in FIG. 3A. The same or a different clinician can administer the next injection—which can be days, weeks, or even months later—using a locator and needle guide that is substantially identical to locator and needle guide 300A and rotated by a given angle (e.g., 45 degrees clockwise or counterclockwise) relative to the orientation of locator and needle guide 300A during the initial injection. This process can be iterated for each subsequent injection to ensure that the septum of the subcutaneous structure is pierced by the needle at different locations. It will be appreciated, however, that it can be unlikely for the needle to pierce the septum in the same location even without a clinician deliberately rotating locator and needle guide 300A during subsequent injections.

With reference to FIG. 3B, shown is a top view of locator and needle guide 300B. Locator and needle guide 300B includes base portion 310B and needle guide portion 320B. Base portion 310B can be similar to base portion 210. Locator and needle guide 300B can be similar to locator and needle guide 115, except that needle guide portion 320B defines apertures 330B, each configured to have a corresponding needle inserted there through and align the corresponding needle at a target angle relative to the subcutaneous structure. Although apertures 330B comprise five apertures arranged in a line, it will be appreciated that the locator and needle guides described herein can define any suitable number of apertures in any suitable arrangement.

Like locator and needle guide 300A, locator and needle guide 300B can facilitate repeatable injections at the same site or at different sites on the septum (and/or into multiple septa close to each other). Locator and needle guide 300B can also/alternatively be used to facilitate injecting different therapeutic substances at different locations in a subcutaneous structure. For example, if the subcutaneous structure has multiple septa, locator and needle guide 300B can be used to inject each septum with a respective therapeutic substance. More specifically, the clinician can insert multiple needles into needle guide portion 320B to align each of the multiple needles at the target angle relative to the subject and administer multiple injections into multiple compartments of the subcutaneous structure. In some examples, base portion 310B may include an orientation feature configured to fixedly orient/mate base portion 310B with the subcutaneous structure (and the multiple compartments).

With reference to FIG. 3C, shown is a cross-sectional view 300C of aperture 310C and needle 320C inserted there through. Aperture 310C can be defined by a needle guide of a locator and needle guide in accordance with techniques described herein. As shown, an inner dimension of aperture 310C (e.g., diameter) is wider than an outer dimension (e.g., diameter) of needle 320C. This arrangement permits needle 320C (or a different respective needle) to be inserted at a different location of the septum for each subsequent injection, while still ensuring that needle 320C enters the septum at the target angle (e.g., within a range of acceptable angles). In some examples, the needle guide may further define a frustoconical shape where needle 320C enters aperture 310C to improve ease of needle insertion.

With reference to FIG. 3D, shown is a cross-sectional view 300D of aperture 310D and needle 320D inserted there through. Aperture 310D can be defined by a needle guide of a locator and needle guide in accordance with techniques described herein. As shown, aperture 310D has a frustoconical shape. Like in FIG. 3C, this arrangement permits needle 320D (or a different respective needle) to be inserted at a different location of the septum for each subsequent injection, while still ensuring that needle 320D enters the septum at the target angle (e.g., within a range of acceptable angles).

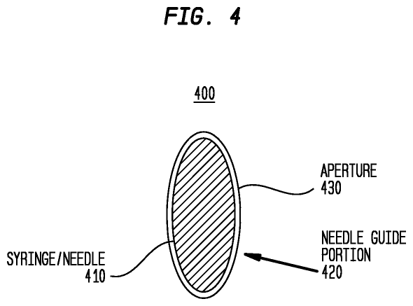

FIG. 4 illustrates a cross-sectional view 400 of needle (and/or syringe connected to thereto) 410 and needle guide portion 420 that are uniquely compatible with each other, in accordance with certain embodiments presented herein. Needle guide portion 420, which can be part of a locator and needle guide described herein, defines aperture 430 through which needle/syringe 410 is inserted. Aperture 430 is uniquely compatible with a specific needle/syringe type of needle/syringe 410. Administering an incorrect therapeutic substance can have catastrophic consequences for the patient, and this feature can ensure that the correct therapeutic substance is administered (e.g., in case the specific needle/syringe type of needle/syringe 410 is assigned to a specific therapeutic substance).

In the example of FIG. 4, an inner dimension or shape of aperture 430 (here, oval) is uniquely compatible with an outer dimension or shape of the specific needle/syringe type of needle/syringe 410. If the clinician attempts to administer an injection with a different therapeutic substance/needle type, the inner dimension or shape of needle guide portion 420 acts as a hard stop to prevent needle/syringe 410 from entering the patient. For example, if the needle with the wrong therapeutic substance has a circular shape, it cannot fit in aperture 430, which has an oval shape. Thus, the mismatch can prevent the clinician from mistakenly administering the wrong therapeutic substance. It will be appreciated that the needle and aperture can be any suitable shape and/or dimensions to ensure the correct therapeutic substance is administered.

In one example, the base portions and needle guide portions described herein can be provided as one integrated apparatus (e.g., inherent to the same part) or configured for assembly (e.g., snapping or stacking) via respective mating components/features. In either case, the needle guide portion can be designed to uniquely fit the syringe. If a base portion and needle guide portion are provided as one integrated apparatus, a specific diameter or shape of an axially oriented guiding feature (e.g., cylindrical, etc.) inherent to the locator and needle guide can mate with a specific diameter or shape of at least a portion of the syringe/needle (e.g., the needle itself and/or a container portion of the syringe configured to hold the therapeutic substance, such as a plastic container portion or feature (e.g., an add-on feature) applied during syringe manufacturing).

Or, if the base portion and needle guide portion are configured for assembly, a specific diameter or shape of a first axially oriented guiding feature (e.g., cylindrical, etc.) inherent to the needle guide portion can mate with a specific diameter or shape of at least a portion of the syringe/needle (e.g., the needle itself and/or a container portion of the syringe configured to hold the therapeutic substance, such as a plastic container portion). The specific diameter or shape of a second feature (e.g., cylindrical, etc.) of the needle guide portion can mate with a feature (e.g., cylindrical, etc.) of the locator portion. It will be appreciated that the needle guide portion and/or locator portion can include any suitable number of complementary mating features (e.g., as a feature set).

In one example, the locator and needle guide can include a lock (e.g., a snap) configured to trap the syringe in the apparatus. The lock can prevent the syringe from being separated from the locator and needle guide after the syringe is fully inserted into (mated with) the locator and needle guide. The locator and needle guide (or the needle guide portion) and the syringe can be supplied together. The syringe and/or locator and needle guide can have a specific color and/or shape that corresponds to a specific therapeutic substance type, which can explicitly signal to the surgeon/clinician which type of therapeutic substance is to be used.

A needle may be positioned in any suitable manner with a needle guide portion. As described above, in one example, the needle may be inserted into a needle guide portion. However, in other examples, the needle guide portion may serve as a visual/partial/reference guide. For example, the needle guide portion may define a hollow/cavity in the shape of a half cylinder truncated longitudinally configured to guide a syringe/needle.

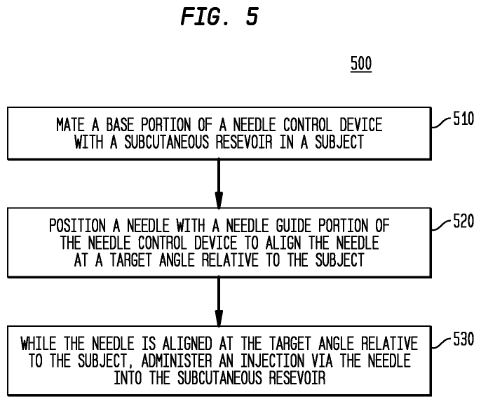

FIG. 5 is a flowchart of a method 500, in accordance with certain embodiments presented herein. Method 500 is a method for administering an injection using a locator and needle guide described herein. At operation 510, a base portion of a needle control device is mated with a subcutaneous reservoir in a subject. At operation 520, a needle is positioned with a needle guide portion of the needle control device to align the needle at a target angle relative to the subject. At operation 530, while the needle is aligned at the target angle relative to the subject, an injection is administered via the needle into the subcutaneous reservoir.

As previously noted, the locator and needle guides described herein may be used to make a mark (e.g., a temporary or permanent tattoo) on the skin of the patient over a subcutaneous structure. For instance, a locator and needle guide can guide a marking device (e.g., a tattoo needle) used to place a temporary/initial mark on the skin. In one example, the mark may be placed directly above/over the position of the subcutaneous structure. In another example, the mark may have a predefined position relative to the subcutaneous structure. In any case, the mark may indicate a position on the subject where the subcutaneous structure is located. A specific target angle (e.g., 90-degrees) may not be necessary when making the temporary mark. A permanent mark (e.g., tattoo) can be made based on the temporary/initial mark. In certain examples, the locator and needle guide can be removed after the temporary mark is made, and the permanent mark can be made based on the location of the initial mark without the locator and needle guide. A clinician can use the permanent mark to locate and replenish the subcutaneous structure during iterative needle insertions/therapeutic substance delivery cycles. The clinician can also use the locator and needle guide described herein to administer the subsequent injections.

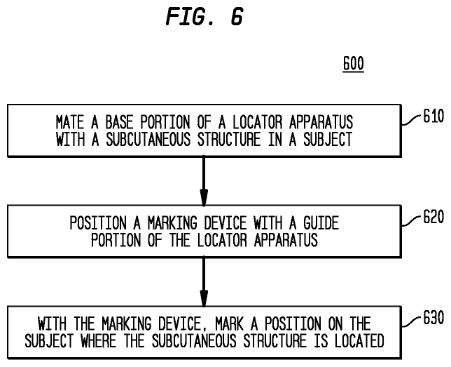

FIG. 6 is a flowchart of another method 600, in accordance with certain embodiments presented herein. Method 600 is a method for marking a position on a subject using a locator apparatus. At operation 610, a base portion of a locator apparatus mates with a subcutaneous structure in a subject. At operation 620, a marking device is positioned with a guide portion of the locator apparatus. At operation 630, a position on the subject where the subcutaneous structure is located is marked with the marking device. In a further example, in response to the position being marked, the position can be tattooed (e.g., temporarily, semi-permanently, or permanently marked).

As previously described, the technology disclosed herein can be applied in any of a variety of circumstances and with a variety of different devices. The techniques of the present disclosure can be applied to implantable stimulation systems, neurostimulators, cardiac pacemakers, cardiac defibrillators, sleep apnea management stimulators, seizure therapy stimulators, tinnitus management stimulators, and vestibular stimulation devices, retinal prostheses, as well as other medical devices that deliver stimulation to tissue. Further, technology described herein can also be applied to consumer devices. These different systems and devices can benefit from the technology described herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. An apparatus comprising:
a locator portion configured to locate a target structure disposed in a body of a subject; and
a needle guide portion defining an aperture configured to have a needle positioned therewith, wherein the needle guide portion is configured to align the needle at a target angle relative to the target structure.

2. The apparatus of claim 1, wherein the aperture is centered with respect to the needle guide portion.

3. The apparatus of claim 1, wherein the aperture is off-center with respect to the needle guide portion.

4. The apparatus of claim 1, wherein the needle guide portion defines a plurality of apertures each configured to have a corresponding needle positioned therewith, wherein each of the plurality of apertures is configured to align the corresponding needle at a target angle relative to the target structure.

5. The apparatus of claim 1, wherein an inner dimension of the aperture is wider than an outer dimension of the needle.

6. The apparatus of claim 5, wherein the aperture has a frustoconical shape.

7. (canceled)

8. (canceled)

9. The apparatus of claim 1, wherein the target angle is between 70 and 110 degrees.

10. The apparatus of claim 9, wherein the target angle is between 80 and 100 degrees.

11. The apparatus of claim 10, wherein the target angle is between 85 and 95 degrees.

12. The apparatus of claim 1, wherein the locator portion is configured to mate with the target structure.

13. The apparatus of claim 12, wherein the target structure is a subcutaneous reservoir comprising a skin-facing membrane, and wherein the needle guide portion is configured align the needle at the target angle relative to the skin-facing membrane.

14. The apparatus of claim 13, wherein the locator portion is shaped to be disposed around an outer dimension of the subcutaneous reservoir and is sized to ensure that the needle positioned with the needle guide portion punctures the skin-facing membrane.

15. The apparatus of claim 14, further comprising a flexible layer affixed to the locator portion and configured to adapt to the outer dimension of the subcutaneous reservoir.

16. The apparatus of claim 1, wherein the locator portion and the needle guide portion are configured for assembly via respective mating components and/or mating features.

17. (canceled)

18. An apparatus comprising:
a base portion configured to mate with a subcutaneous structure; and
a handle portion that is secured to, and elevated from, the base portion, wherein the handle portion is configured to allow a needle to pass there through to the subcutaneous structure.

19. The apparatus of claim 18, wherein the base portion is shaped to be disposed around an outer dimension of the subcutaneous structure.

20. The apparatus of claim 18, wherein the base portion has an inner dimension configured to accommodate both the subcutaneous structure and tissue of a subject in which the subcutaneous structure is implanted when the base portion mates with the subcutaneous structure.

21. The apparatus of claim 18, wherein the base portion has a ring shape.

22. The apparatus of claim 18, wherein the base portion has an adjustable inner dimension.

23. The apparatus of claim 18, wherein the handle portion is elevated from the base portion by an adjustable height.

24. A method comprising:
mating a base portion of a needle control device with a subcutaneous reservoir in a subject;
positioning a needle with a needle guide portion of the needle control device to align the needle at a target angle relative to the subject; and
while the needle is aligned at the target angle relative to the subject, administering an injection via the needle into the subcutaneous reservoir.

25. The method of claim 24, wherein mating the base portion with the subcutaneous reservoir includes:
pressing the base portion to the subject.

26. The method of claim 24, wherein mating the base portion with the subcutaneous reservoir includes:
adjusting an inner dimension of the base portion.

27. The method of claim 24, wherein:
positioning the needle comprises positioning multiple needles with the needle guide portion to align each of the multiple needles at the target angle relative to the subject; and
administering the injection via the needle into the subcutaneous reservoir comprises administering multiple injections into multiple compartments of the subcutaneous reservoir.

28. The method of claim 24, further comprising:
adjusting an elevation of the needle guide portion from the base portion.

29. (canceled)

30. (canceled)

31. (canceled)

32. (canceled)
